(19) United States
(12) Patent Application Publication
(10) Pub. No.: US 2025/0161688 A1
(43) Pub. Date: May 22, 2025

(54) Systems and Methods for Randomized Coordinated Reset Deep Brain Stimulation

(71) Applicant: The Board of Trustees of the Leland Stanford Junior University, Stanford, CA (US)

(72) Inventors: Peter A. Tass, Palo Alto, CA (US); Ali Khaledi Nasab, San Jose, CA (US)

(73) Assignee: The Board of Trustees of the Leland Stanford Junior University, Stanford, CA (US)

(21) Appl. No.: 18/833,852

(22) PCT Filed: Jan. 27, 2023
(86) PCT No.: PCT/US2023/061508

§ 371 Date: Jul. 26, 2024

Related U.S. Application Data
(60) Provisional application No. 63/267,290, filed on Jan. 28, 2022.

(51) Int. Cl. A61N 1/36; A61N 1/05

(52) U.S. Cl. CPC A61N 1/36167; A61N 1/0534; A61N 1/36067

(57) ABSTRACT

Systems and methods for randomized coordinated reset deep brain stimulation in accordance with embodiments of the invention are illustrated. One embodiment includes a deep brain stimulation system and a neurostimulator coupled to a plurality of electrodes, where the neurostimulator is configured to deliver randomized coordinated reset stimulation to a brain via the plurality of electrodes, where the coordinated reset stimulation comprises a plurality of consecutive time windows, where within each consecutive time window, a pulse is delivered by each electrode in the plurality of electrodes at a respective amplitude at a respective time in the time window, and wherein at least one of: the respective amplitude of the pulse for a given electrode in the plurality of electrodes is randomly varied in each consecutive time window, and the respective time of the pulse for a given electrode in the plurality of electrodes is randomly varied in each consecutive time window.

### CROSS-REFERENCE TO RELATED APPLICATIONS

The current application is a U.S. National Stage Patent Application of PCT Patent Application No. PCT/US2023/061508 entitled “Systems and Methods for Randomized Coordinated Reset Deep Brain Stimulation” filed Jan. 27, 2023, which claims the benefit of and priority under 35 U.S.C. § 119(e) to U.S. Provisional Patent Application No. 63/267,290 entitled “Systems and Methods for Randomized Coordinated Reset Deep Brain Stimulation” filed Jan. 28, 2022. The disclosures of PCT Patent Application No. PCT/US2023/061508 and U.S. Provisional Patent Application No. 63/267,290 are hereby incorporated by reference in its entirety for all purposes.

### FIELD OF THE INVENTION

The present invention generally relates to deep brain stimulation, and more specifically, delivery of randomized coordinated reset therapy via deep brain stimulation.

### BACKGROUND

Parkinson's disease (PD) is a degenerative disorder of the central nervous system that is marked by four cardinal motor symptoms: bradykinesia, rigidity, tremor, and postural instability. Typically, PD symptoms are managed with medication (primarily L-DOPA). However, as the disease progresses, medications become less effective and can begin to have adverse side effects. In drug-resistant patients, an invasive surgery to place microelectrodes intro the brain for the delivery of deep brain stimulation is performed. A neurostimulator delivers electrical impulses through the electrodes into the brain in order to alleviate the motor symptoms. DBS is also used for the treatment of obsessive-compulsive disorder, and epilepsy, among others.

### SUMMARY OF THE INVENTION

Systems and methods for randomized coordinated reset deep brain stimulation in accordance with embodiments of the invention are illustrated. One embodiment includes a deep brain stimulation system, including a plurality of electrodes, and a neurostimulator coupled to the plurality of electrodes, where the neurostimulator is configured to deliver randomized coordinated reset stimulation to a brain via the plurality of electrodes, where the coordinated reset stimulation comprises a plurality of consecutive time windows, where within each consecutive time window, an electric pulse is delivered by each electrode in the plurality of electrodes at a respective amplitude at a respective time in the time window, and wherein at least one of: the respective amplitude of the electric pulse for a given electrode in the plurality of electrodes is randomly varied in each consecutive time window, and the respective time of the electric pulse for a given electrode in the plurality of electrodes is randomly varied in each consecutive time window.

In another embodiment, the variance of the respective amplitude of the electric pulse for the given electrode is varied using a uniform distribution

In a further embodiment, the variance of the respective amplitude of the electric pulse for the given electrode is varied using a binary distribution

In still another embodiment, the respective time in the time window for the given electrode is randomly varied.

In a still further embodiment, both the respective amplitude and the respective time of the electric pulse for the given electrode is randomly varied.

In yet another embodiment, the respective amplitude of the electric pulses for all electrodes in the plurality of electrodes is randomly varied in each consecutive time window, and the respective amplitude of the electric pulses for all electrodes in the plurality of electrodes is randomly varied in each consecutive time window

In a yet further embodiment, the randomized coordinated reset stimulation treats the motor symptoms of Parkinson's Disease.

In another additional embodiment, a method of deep brain stimulation, including delivering randomized coordinated reset stimulation to a brain via a plurality of electrodes, where the coordinated reset stimulation comprises a plurality of consecutive time windows, where within each consecutive time window, an electric pulse is delivered by each electrode in the plurality of electrodes at a respective amplitude at a respective time in the time window, and wherein at least one of: the respective amplitude of the electric pulse for a given electrode in the plurality of electrodes is randomly varied in each consecutive time window, and the respective time of the electric pulse for a given electrode in the plurality of electrodes is randomly varied in each consecutive time window.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 illustrates example randomized coordinated reset patterns in accordance with an embodiment of the invention.

FIG. 2 is a system diagram for a neurostimulation system that uses randomized coordinated reset in accordance with an embodiment of the invention.

FIG. 3 is a block diagram for a neurostimulator in accordance with an embodiment of the invention.

FIG. 4 is a flow chart for a neurostimulation using randomized coordinated reset in accordance with an embodiment of the invention.

### DETAILED DESCRIPTION

Abnormal synchrony of neurological activity is related to several brain disorders, including Parkinson's Disease and epilepsy. Invasive stimulation techniques of the brain or the spinal cord are often used to counteract abnormal neuronal activity in severe cases. Conventional deep brain stimulation (DBS) protocols involve periodically delivering charge-balanced electrical pulses at sufficiently high rates, typically greater than 100 Hz (so-called high-frequency stimulation) to suppress symptoms via electrodes implanted in the brain. Delivering pulses at lower rates, e.g., 20 Hz, may strongly evoke or enhance symptoms. Typically, both amplitudes of electrical pulses and pulse rate of standard DBS are kept constant. DBS leads (electrodes) typically comprise several stimulation contacts of different shape, e.g., ring-type or segmented contacts. Optimal stimulation contacts are typically selected by clinical testing, weighing acute therapeutic effects and side effects. However, these techniques may cause side effects due to unwanted stimulation of tissue adjacent to the target area, e.g., mediated by current spread or due to stimulation of fibers passing through the target area. Stimulation of adjacent tissue is more likely in the case of sub-optimal placement of stimulation electrodes. Even with optimal placement, stimulation of fibers passing through a target area is often inevitable. Accordingly, to avoid side effects, it is desirable to induce sufficient stimulation effects with minimal integral stimulation current.

To this end, stimulation techniques were developed which cause long-lasting, sustained effects that persist after cessation of stimulation. A key technique in this context is Coordinated Reset (CR) stimulation, which was designed to treat abnormal neuronal synchrony, aiming at an unlearning of the underlying abnormal synaptic connectivity, which ultimately results in long-lasting desynchronization. However, CR stimulation involves adjustment of different stimulation parameters, in particular, a stimulation frequency (a repetition rate of applied stimulation bursts) to a dominant frequency of an abnormal neuronal synchronized oscillation. If not properly adjusted, inappropriate values of the stimulation frequency can cause resonance-like effects, resulting in strong and unwanted increase of neural synchrony. In addition, CR stimulation involves proper selection of different (adequately spaced) stimulation sites within a tissue volume, which imposes significant calibration demands. In addition, due to sub-optimal placement of implanted depth electrodes, it may be difficult to find more than one stimulation contact (for unipolar stimulation), or more than one pair of stimulation contacts (for bipolar stimulation). In these cases, multi-site stimulation algorithms, such as CR stimulation, cannot be applied.

Proper parameter calibration and adjustment of DBS techniques and, especially, multi-channel stimulation algorithms such as CR stimulation, can be challenging and, in turn, limit therapeutic efficacy, since: (i) system parameters, representing characteristics of the neuronal tissue (e.g. activity and connectivity) typically vary in time; (ii) several brain disorders, such as Parkinson's disease, are characterized by the presence of multiple abnormal rhythms with different frequencies which hinder the adjustment of the stimulation frequency; (iii) relevant quantities, such as the strength of the synaptic connectivity, cannot be assessed sufficiently; and (iv) the relationship of the targets activity and connectivity and its long-term responses to available stimulus techniques may be intricate, reflecting complex, non-linear dependencies.

Systems and methods for deep brain stimulation described herein can address the problems listed above in both single-site and multi-site stimulation scenarios. Variants of CR are described which randomly vary one or both of two key stimulation parameters: stimulus amplitude and delivery time. In many embodiments, for single-site scenarios, instead of delivering standard high-frequency stimulation (at rates typically greater than 100 Hz) to one stimulation contact (monopolar stimulation) or to a contact pair (bipolar stimulation), trains of charge-balanced electrical pulses with randomly varying amplitude are delivered. In various embodiments, a single pulse is used instead of a pulse train per time window. Randomization of pulse amplitudes can be obtained in different ways, i.e., pulse amplitude randomization can be realized by means of different random processes or deterministic, e.g., chaotic processes or combinations thereof. In many embodiments, robust effects can be achieved by simply using a randomized amplitude with uniform probability distribution. in the interval ranging from 0 to Aₘₐₓ, where Aₘₐₓ denotes the maximum amplitude. A discussion of determination Aₘₐₓ is found further below. Amplitudes, and, in general, the administered current has to fulfill safety standard requirements preventing from tissue damage. These safety requirements are fulfilled by implantable pulse generators approved for clinical use. Single-site stimulation is performed if pronounced acute effects can only or predominately be achieved with only one contact (for unipolar stimulation) or one contact pair (for bipolar stimulation).

Amplitude randomization can be applied to different temporal stimulation patterns. Let us denote the times at which single charge-balanced electrical pulses are delivered by t₁, t₂, t₃, . . . and the corresponding amplitudes of these pulses by A₁, A₂, A₃, . . . For example, in many embodiments, a periodic pulse train with dₜ=t₂−t₁=t₃−t₂=. . . and amplitudes A₁, A₂, A₃, . . . randomly varying from one pulse to another is used. However, in general, stimulation can also be varied in a deterministic, randomized or chaotic manner or combinations thereof. For instance, denoting the mean amplitude of pulse delivery by Tₘ, within each stimulation period of length Tₘ one stimulus is delivered, where the exact stimulus time varies randomly within the interval between Tₘ and 2*Tₘ with uniform probability. To desynchronize a wide range of abnormal neuronal rhythms, the pulse train frequency 1/dₜ or mean pulse train frequency should be chosen to exceed the lowest frequency of abnormal rhythms by a factor of 3 up to a factor of 30 or even 50 or more, in this way effectively desynchronizing a large spectrum of abnormal neuronal rhythms.

For multisite stimulation, if acute stimulation effects can be achieved through more than one stimulation contact (in unipolar stimulation mode) or more than one pair of stimulation contacts (in bipolar stimulation mode), then the process described above with respect to single-site stimulation can be applied for different channels in an uncorrelated manner to achieve improved results. As used herein, a channel is either one stimulation contact activated in unipolar mode or a pair of stimulation contacts activated in bipolar mode. For illustration, for two-channel stimulation the stimulation times and corresponding stimulation amplitudes for channel a read t₁a, t₂a, t₃a, . . . and A₁a, A₂a, A₃a, . . . , whereas stimulation times and corresponding stimulation amplitudes for channel b are given by t₁b, t₂b, t₃b, . . . and A₁b, A₂b, A₃b, . . . . Denote the randomization processes generating t₁a, t₂a, t₃a, . . . , t₁b, t₂b, t₃b, . . . , A₁a, A₂a, A₃a, . . . , and A₁b, A₂b, A₃b, . . . . By Rta, Rtb, RAa, and RAb, respectively. In many embodiments, all four random processes, i.e. Rta, Rtb, RAa, and RAb, are uncorrelated to achieve optimal results. Definitions of different classes of randomized CR are discussed below.

### Randomized CR Paradigms

The regular CR stimulation pattern is characterized by the stimulation frequency, f_{CR}, which sets the CR cycle period 1/f_{CR}, and the number of separately stimulated subpopulations, Nₛ. During each CR cycle, each subpopulation receives exactly one stimulus. Obeying this restriction, individual stimuli are delivered at subsequent multiples of 1/Nₛf_{CR} to random subpopulations. As noted above, randomized CR refers to variants of CR where the stimulation pulses are randomly varied in a time window and/or in amplitude. In many embodiments, the amplitudes are varied in accordance with a binary distribution. In various embodiments, the amplitudes are varied in accordance with a uniform distribution. This gives rise to 5 randomized CR paradigms: temporally uncorrelated CR (tCR), CR with binary distributed stimulus amplitudes (bCR), CR with uniformly distributed stimulus amplitudes (uCR), and two variants of double random (time and amplitude) CR stimulation btCR, and utCR.

tCR is similar to CR stimulation, however each subpopulation receives exactly one stimulus per cycle at a uniformly distributed time between zero and 1/f_{CR}. In many embodiments, there is no correlation between the stimulus times of different channels. bCR and uCR are also similar to CR stimulation, but the amplitude of each stimulus is randomly chosen according to either a binary distribution or a uniform distribution, respectively. For uCR, stimulus amplitudes are uniformly distributed between Aₛₜᵢₘ=0 and Aₛₜᵢₘ=1. For Aₛₜᵢₘ=1, the integral current over the excitatory part of a single stimulus is strong enough to drive neuron membrane potential over the spiking threshold. In various embodiments, for uCR, stimulus amplitudes are uniformly distributed between between Aₛₜᵢₘ=0 and Aₛₜᵢₘ=Aₘₐₓ. For bCR, stimuli possess amplitudes of either Aₛₜᵢₘ=0 or Aₛₜᵢₘ=1. In some embodiments, for bCR, stimuli possess amplitudes of either Aₛₜᵢₘ=0 or A_{ben}<Aₛₜᵢₘ<Aₘₐₓ. This corresponds to the removal of a random fraction 1−p of pf stimuli from the pattern, where p is the fraction of delivered stimuli. The probability p at which stimuli have a non-zero Aₛₜᵢₘ is a free parameter. Double random CR stimulation combines tCR with either bCR or uCR, yielding btCR and utCR, where the stimulation pattern has randomized stimulus times and randomized stimulus amplitudes.

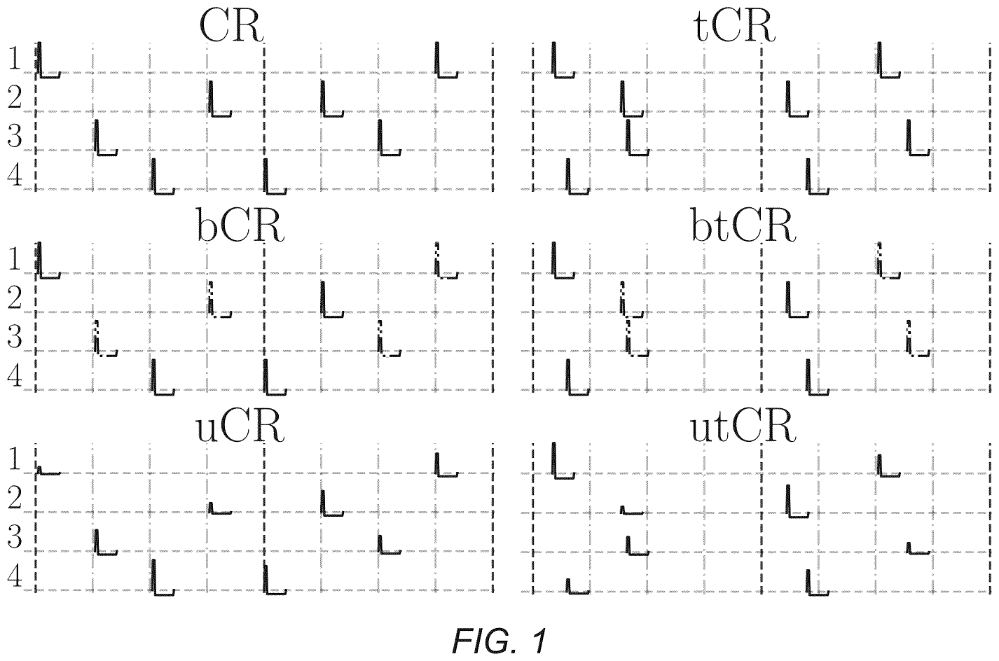

Turning now to FIG. 1, example stimulation patterns for CR and the 5 variants of randomized CR are illustrated in accordance with an embodiment of the invention for comparison. While four subpopulations are shown in the charts, as can readily be appreciated, the number of subpopulations can be increased or decreased as appropriate to the requirements of specific applications of embodiments of the invention. Dashed vertical lines indicate the start of a new stimulation cycle. Dashed pulses in bCR and btCR that are removed due to Aₛₜᵢₘ=0. Pulses on the same row are delivered to the same subpopulation.

All variants of randomized CR can be personalized to a patient by a calibration process which tunes the pulse widths, amplitude ranges, and/or pulse frequency. Patients can be given test stimuli and a safe and therapeutic range for a given patient can be determined. For example, a single stimulus pulse is parameterized by pulse width, pulse amplitude, and pulse frequency. In numerous embodiments, the pulse width (i.e., duration of single stimulus pulse) is chosen in the range 30 μs-210 μs. However, even shorter pulse widths, e.g., down to 20 μs are possible depending on the response of the patient.

With respect to pulse amplitude, the “therapeutic window” is a term referring to standard high-frequency DBS. It is defined as the interval ranging from the minimal pulse amplitude, A_{ben}, required to produce a beneficial effect, up to the minimum pulse amplitude, A_{adv}, required to produce adverse effects. In many embodiments, the therapeutic window for each stimulation contact (monopolar stimulation) or pair of stimulation contacts (bipolar stimulation) for a given patient is experimentally determined. For instance, the therapeutic window should be separately determined for each segment (i.e., stimulation contact) of a segmented electrode. For the assessment of the therapeutic window, test pulse amplitudes are slowly and gradually increased while maintaining a defined and constant pulse width and stimulation frequency. In many embodiments, during this calibration test, either standard high-frequency stimulation or a single channel pulse train stimulation subject to temporal jitter is used. In many embodiments, the therapeutic window changes with pulse width and stimulation frequency.

In various embodiments, the pulse amplitude is gradually increased during calibration in steps of 0.1-0.5 V or 0.1-0.5 mA up to a maximum of 5 V or 5 mA (for voltage and current-controlled implantable pulse generators) and beneficial effects (in particular, on quickly responding symptoms like tremor and rigidity) are observed to determine A_{ben}. The increase of pulse amplitude is stopped at A_{adv}, i.e., as soon as side effects occur. If A_{ben} is not required (see below), the focus is on the occurrence of side effects. This is often easier since side effects tend to build up rapidly, whereas beneficial effects on symptoms other than tremor may take some time.

In many embodiments, Aₘₐₓ (the maximum stimulation amplitude) is set to c·A_{adv}, where c is a constant ≤1, typically in the range 0.7≤c≤1, often 0.8≤c<1 to make sure stimulation cannot elicit side effects. In various embodiments, if the beneficial effects are not pronounced, e.g., due to suboptimal electrode placement, c can attain values greater than 1. This can be tested by gradually increasing the pulse amplitude in steps of 0.1-0.5 V or 0.1-0.5 mA while using the selected randomized CR pattern (rather than the standard high-frequency) stimulation. In some embodiments, in uCR and utCR, the pulse amplitude is randomized in the interval ranging from 0 to Aₘₐₓ, with uniform probability distribution. In numerous embodiments, in uCR and utCR, the pulse amplitude is randomized in the interval ranging from A_{ben} to Aₘₐₓ, with uniform probability distribution. In the second case, A_{ben} serves as an effective zero.

With respect to stimulation frequency, standard high-frequency DBS, stimulation frequencies are typically greater than 100 Hz, often around 130 Hz and often below 185 Hz, and rarely smaller than 100 Hz, e.g., reaching down to 33 Hz. In contrast, randomized CR can utilize stimulation frequencies in the entire range between 1 Hz up to 200 Hz and up to 300 Hz or more. If beneficial effects are not seen using a single pulse, they can be replaced by a by a burst, i.e., by a group of a few identical pulses with sufficiently high intra-burst frequency, e.g., the bursts are delivered with a (mean) frequency in the range between 1 Hz and 30 Hz, whereas the intra-burst frequency ranges between 100 Hz and 300 Hz or more.

As can be readily appreciated, pulse amplitude ranges, pulse frequency, and pulse width may vary depending on the individual patient. In many embodiments, these numbers are tuned to the patient experimentally, and while the above reflects typical ranges, specific patients may require different pulse parameters. Irrespective of pulse parameters, randomized CR patterns of said pulses can provide increased treatment efficacy as compared to delivery in accordance with conventional CR. Delivery of randomized CR is discussed below.

### Delivery of Randomized CR

Randomized CR patterns can be delivered to patients using neurostimulators such as those typically used for deep brain stimulator. In many embodiments, the neurostimulator is an implanted device with leads to electrodes in the brain. The neurostimulator can be programmed using an external device with stimulation patterns and/or activation times. In various embodiments, the neurostimulator can be triggered via a programming device.

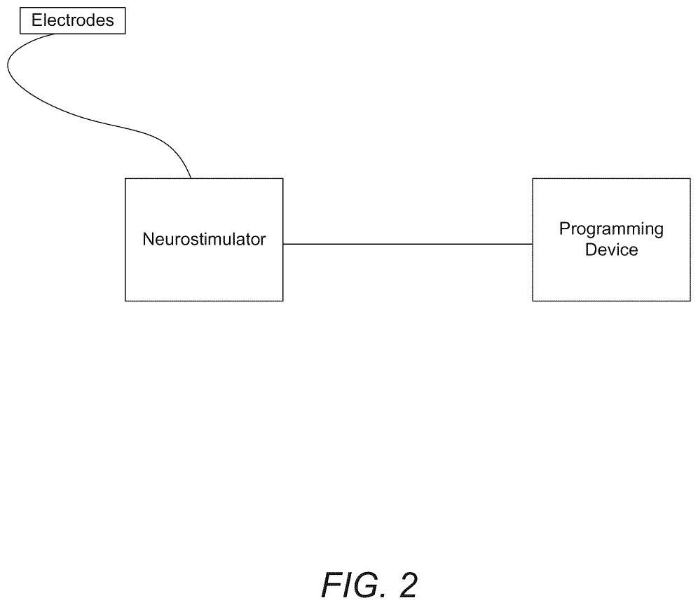

Turning now to FIG. 2, a neurostimulation system in accordance with an embodiment of the invention is illustrated. Neurostimulation system 200 includes a neurostimulator 210 which can be implanted into a patient, and electrodes 220 which are designed to be implanted into the patient's brain. The neurostimulation system 200 further includes a programming device 230 which can be used to program the neurostimulator. In numerous embodiments, the programming device communicates wirelessly with the neurostimulator. The programming device can be used to select a randomized coordinated reset pattern for delivery via the electrodes. While a particular system is shown in FIG. 2, as can be readily appreciated any number of architectures can be used such as (but not limited to) those that utilize other programming mechanisms.

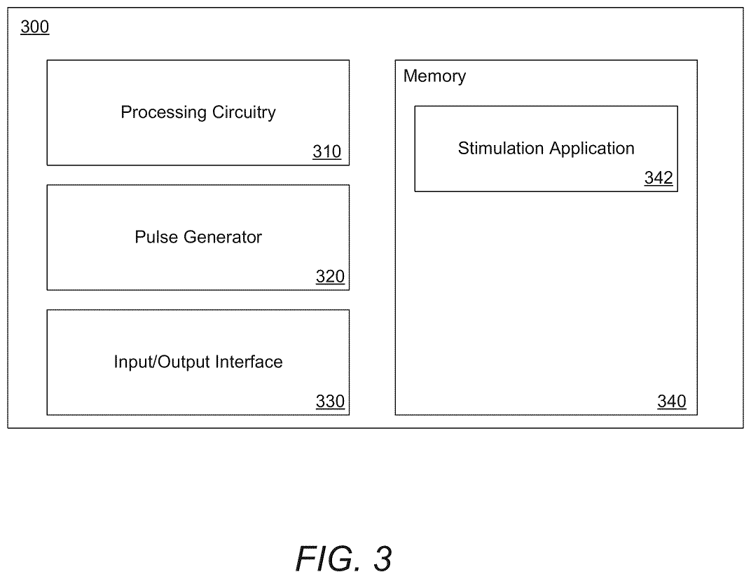

A block diagram for a neurostimulator in accordance with an embodiment of the invention is illustrated in FIG. 3. Neurostimulator 300 includes a processing circuitry 310 which controls the performance of the pulse generator 320. The pulse generator 320 is capable of generating stimulation pulses and delivering them via connected electrodes to the brain. The neurostimulator 300 further includes an input/output interface 330 for communicating with programming devices, and a memory 340. The memory 340 contains a stimulation application which configures the processing circuitry to generate pulses in accordance with a selected randomized CR pattern. The memory can be volatile memory, non-volatile memory, and/or any combination thereof. The processing circuitry can be any logic circuit capable of controlling the pulse generator including (but not limited to) a microprocessor, an application-specific integrated circuit, a field-programmable gate array, and/or any other processing device as appropriate to the requirements of specific applications of embodiments of the invention.

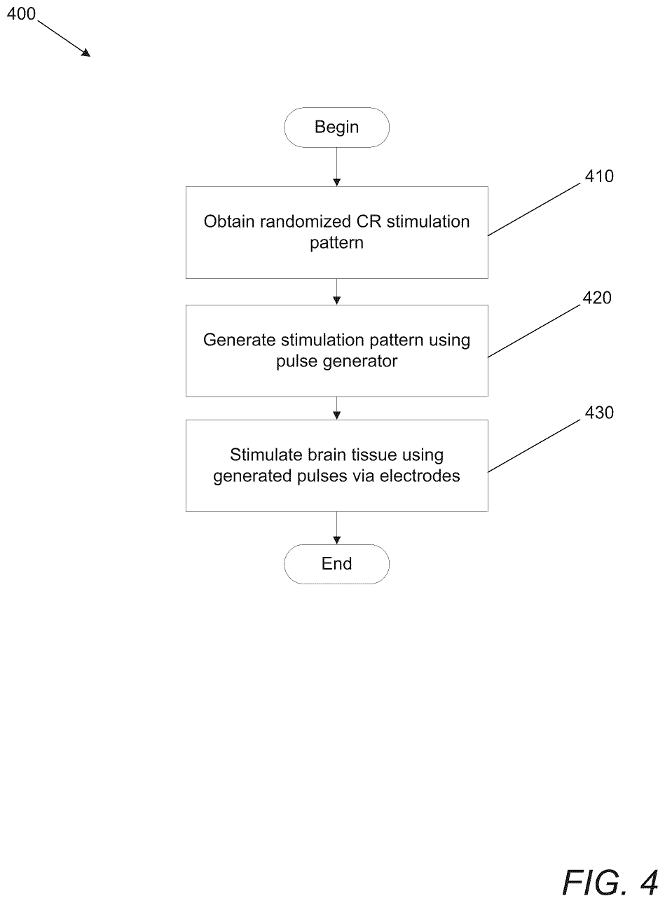

Turning now to FIG. 4, a flow chart for delivering randomized CR in accordance with an embodiment of the invention is illustrated. Process 400 includes obtaining (410) a randomized CR pattern from a programming device. In many embodiments, the programming device indicates which randomized CR pattern the neurostimulator should use from a library of patterns already stored on the neurostimulator. In various embodiments, the neurostimulator is provided with a randomized CR pattern by the programming device. The randomized CR stimulation pattern is generated (420) by the pulse generator and the target brain tissue is stimulated (430) using the generated pulses.

Although specific systems and methods for deep brain stimulation using randomized coordinated reset are discussed herein, many different methods can be implemented in accordance with many different embodiments of the invention. For example, different amplitude ranges, timing patterns, and/or methods to introduce randomness can be used as appropriate to the requirements of specific applications of embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A deep brain stimulation system, comprising:
a plurality of electrodes; and
a neurostimulator coupled to the plurality of electrodes;
where the neurostimulator is configured to deliver randomized coordinated reset stimulation to a brain via the plurality of electrodes;
where the coordinated reset stimulation comprises a plurality of consecutive time windows;
where within each consecutive time window, an electric pulse is delivered by each electrode in the plurality of electrodes at a respective amplitude at a respective time in the time window; and
wherein at least one of:
the respective amplitude of the electric pulse for a given electrode in the plurality of electrodes is randomly varied in each consecutive time window; and
the respective time of the electric pulse for a given electrode in the plurality of electrodes is randomly varied in each consecutive time window.

2. The system of claim 1, wherein the variance of the respective amplitude of the electric pulse for the given electrode is varied using a uniform distribution.

3. The system of claim 1, wherein the variance of the respective amplitude of the electric pulse for the given electrode is varied using a binary distribution.

4. The system of claim 1, wherein the respective time in the time window for the given electrode is randomly varied.

5. The system of claim 1, wherein both the respective amplitude and the respective time of the electric pulse for the given electrode is randomly varied.

6. The system of claim 1, wherein at least one of:
the respective amplitude of the electric pulses for all electrodes in the plurality of electrodes is randomly varied in each consecutive time window; and
the respective amplitude of the electric pulses for all electrodes in the plurality of electrodes is randomly varied in each consecutive time window.

7. The system of claim 1, wherein the randomized coordinated reset stimulation treats the motor symptoms of Parkinson's Disease.

8. A method of deep brain stimulation, comprising:
delivering randomized coordinated reset stimulation to a brain via a plurality of electrodes;
where the coordinated reset stimulation comprises a plurality of consecutive time windows;
where within each consecutive time window, an electric pulse is delivered by each electrode in the plurality of electrodes at a respective amplitude at a respective time in the time window; and
wherein at least one of:
the respective amplitude of the electric pulse for a given electrode in the plurality of electrodes is randomly varied in each consecutive time window; and
the respective time of the electric pulse for a given electrode in the plurality of electrodes is randomly varied in each consecutive time window.

9. The method of claim 8, wherein the variance of the respective amplitude of the electric pulse for the given electrode is varied using a uniform distribution.

10. The method of claim 8, wherein the variance of the respective amplitude of the electric pulse for the given electrode is varied using a binary distribution.

11. The method of claim 8, wherein the respective time in the time window for the given electrode is randomly varied.

12. The method of claim 8, wherein both the respective amplitude and the respective time of the electric pulse for the given electrode is randomly varied.

13. The method of claim 8, wherein at least one of:
the respective amplitude of the electric pulses for all electrodes in the plurality of electrodes is randomly varied in each consecutive time window; and
the respective amplitude of the electric pulses for all electrodes in the plurality of electrodes is randomly varied in each consecutive time window.

14. The method of claim 8, wherein the randomized coordinated reset stimulation treats the motor symptoms of Parkinson's Disease.

15. A method of deep brain stimulation for the treatment of Parkinson's Disease, comprising:
delivering randomized coordinated reset stimulation to a brain via a plurality of electrodes;
where the coordinated reset stimulation comprises a plurality of consecutive time windows;
where within each consecutive time window, an electric pulse is delivered by each electrode in the plurality of electrodes at a respective amplitude at a respective time in the time window;
where the respective amplitude of the electric pulse for a given electrode in the plurality of electrodes is randomly varied in each consecutive time window; and
where the respective time of the electric pulse for a given electrode in the plurality of electrodes is randomly varied in each consecutive time window.
